(19) United States
(12) Patent Application Publication
(10) Pub. No.: US 2026/0170684 A1
(43) Pub. Date: Jun. 18, 2026

(54) Tumor Positioning Method, Electronic Device, and Storage Medium

(71) Applicant: OUR UNITED CORPORATION, Shaanxi (CN)

(72) Inventors: Hao Yan, Shaanxi (CN); Dalin Liu, Shaanxi (CN); Jiuliang Li, Shaanxi (CN)

(21) Appl. No.: 19/423,512

(22) Filed: Dec. 17, 2025

(30) Foreign Application Priority Data
Dec. 18, 2024 (CN) 202411874956.4

(51) Int. Cl. G06T 7/73; A61N 5/10; G06T 7/13; G06T 7/246; G06T 7/33

(52) U.S. Cl. CPC G06T 7/74; A61N 5/1049; G06T 7/13; G06T 7/248; G06T 7/337; A61N 2005/1056; G06T 2207/10081; G06T 2207/10088; G06T 2207/30096; G06T 2207/30241

(57) ABSTRACT

The present disclosure provides a tumor positioning method, a device, and a storage medium. The method includes acquiring a target projection image and a reference image of a target object including a tumor at a target angle; determining a tumor positioning manner corresponding to the target angle according to a type of the target angle; and performing matching processing on the target projection image and the reference image based on the tumor positioning manner corresponding to the target angle, to obtain a position of the tumor in the target projection image.

### CROSS REFERENCE TO RELATED APPLICATION

This application claims priority to Chinese Patent Application No. 202411874956.4, filed Dec. 18, 2024, the disclosure of which is hereby incorporated by reference in its entirety.

### BACKGROUND OF THE INVENTION

### Field of the Invention

The present disclosure relates to the field of medical technologies, and in particular, to the field of radiotherapy technologies, and specifically to a tumor positioning method, a device and a storage medium.

### Description of Related Art

During a radiotherapy process, the accurate processing of tumors is a key factor in ensuring treatment efficacy and patient safety. Especially for tumors that move with respiration, if the tumor cannot be accurately positioned, the radiation may miss the tumor target, resulting in the tumor cells not receiving a sufficient radiation dose and thus not being effectively killed. At the same time, it may also cause unnecessary damage to the surrounding healthy tissues.

### SUMMARY OF THE INVENTION

In a first aspect, the present disclosure provides a tumor positioning method, and the method includes: acquiring a target projection image and a reference image of a target object containing a tumor at a target angle; determining a tumor positioning manner corresponding to the target angle according to a type of the target angle; and performing matching processing on the target projection image and the reference image based on the tumor positioning manner corresponding to the target angle, to obtain a position of the tumor in the target projection image.

In a second aspect, the present disclosure provides a tumor positioning apparatus, and the apparatus includes: a communication unit and a processing unit; the communication unit is configured to, acquire a target projection image and a reference image of a target object containing a tumor at a target angle; determine a tumor positioning manner corresponding to the target angle according to a type of the target angle; and perform matching processing on the target projection image and the reference image based on the tumor positioning manner corresponding to the target angle, to obtain a position of the tumor in the target projection image.

In a third aspect, the present disclosure provides an electronic device and the electronic device includes: a processor and a memory for storing instructions executable for the processor; where the processor is configured to execute the instructions, to implement the tumor positioning method as described in the first aspect and any implementation of the first aspect.

In a fourth aspect, the present disclosure provides a computer readable storage medium and the computer readable storage medium has stored instructions therein, and the instructions, when executed on a terminal, cause the terminal to perform the tumor positioning method as described in the first aspect and any implementation of the first aspect.

In a fifth aspect, the present disclosure provides a computer program product containing instructions, and the computer program product, when executed on a computer, cause the computer to perform the tumor positioning method as described in the first aspect and any implementation of the first aspect.

In a sixth aspect, the present disclosure provides a chip and the chip includes: a processor and a communication interface, the communication interface is coupled with the processor, and the processor is configured to execute a computer program or instructions to implement the tumor positioning method as described in the first aspect and any implementation of the first aspect.

Specifically, the chip provided in the present disclosure further includes a memory, configured to store the computer program or instructions.

It should be noted that, all or part of the computer program or instructions may be stored on a computer readable storage medium. Herein, the computer readable storage medium may be packaged together with the processor of the apparatus, or may be packaged separately with the processor of the apparatus, which is not limited to the present disclosure.

In a seventh aspect, the present disclosure provides a radiotherapy system, and the system includes: an image-guided radiotherapy device, an imaging computer device, a control apparatus, and a respiratory detection apparatus, where the imaging computer device is used to perform the tumor positioning method as described in the first aspect and any implementation of the first aspect.

For the descriptions of the second aspect to the seventh aspect in the present disclosure, reference may be made to the detailed descriptions of the first aspect; and for the beneficial effects of the descriptions of the second aspect to the seventh aspect, reference may be made to the analysis of the beneficial effects of the first aspect, which will not be repeated herein.

In the present disclosure, the name of the above tumor positioning apparatus does not constitute limitations on the devices or functional modules themselves, and in practical implementations, these devices or functional modules may be provided with other names. As long as functions of the various devices or functional modules are similar to those of the present disclosure, they fall within the scope of the claims and their equivalent technologies of the present disclosure.

These or other aspects of the present disclosure will become more readily apparent in the following description.

### BRIEF DESCRIPTION OF THE DRAWINGS

FIG. 1 is a schematic diagram of a scene of a radiotherapy system provided in the embodiments of the present disclosure.

FIG. 2 is a flowchart of a tumor positioning method provided in the embodiments of the present disclosure.

FIG. 3 is a flowchart of another tumor positioning method provided in the embodiments of the present disclosure.

FIG. 4 is a structural schematic diagram of a tumor positioning apparatus provided in the embodiments of the present disclosure.

FIG. 5 is a schematic diagram of a hardware structure of an electronic device, provided by the embodiments of the present disclosure.

### DESCRIPTION OF THE INVENTION

The technical solutions in the embodiments of the present disclosure will be described clearly and completely below in conjunction with the drawings in the embodiments of the present disclosure, and it is obvious that the described embodiments are only a part of the embodiments of the present disclosure, but not all of the embodiments. Based on the embodiments in the present disclosure, all other embodiments obtained by those ordinary skilled without creative efforts shall fall within the protection scope of the present disclosure.

Herein, the term “and/or” merely describes an association relationship of associated objects, and means that there may be three kinds of relationships, and for example, A and/or B may mean three cases: A existing alone, both A and B existing simultaneously, and B existing alone.

The terms such as “first” and “second”, etc., in the description and the drawings of the present disclosure are used to distinguish different objects, or to distinguish between different processings of the same object, rather than to describe a specific order of the objects.

Furthermore, the terms “comprising/including” and “having” and any derivations thereof mentioned in the descriptions of the present disclosure are intended to cover non-exclusive inclusion. For example, a process, method, system, product or device that contains a series of steps or units is not limited to the listed steps or units, but optionally also includes other steps or units that are not listed, or optionally also includes other steps or units inherent to the process, method, product or device.

It should be noted that, in the embodiments of the present disclosure, terms such as “exemplary/exemplarily” or “for example” are used to represent examples, illustrations, or descriptions. Any embodiment or design solution described with “exemplary/exemplarily” or “for example” in the embodiments of the present disclosure should not be construed as preferred or advantageous over other embodiments or design solutions. Rather, the usage of the words, such as “exemplary/exemplarily” or “for example”, is intended to present relevant concepts in a specific manner.

In the description of the present disclosure, unless otherwise specified, “multiple/a/the plurality of” refers to two or more.

During a radiotherapy process, the accurate processing of tumors is a key factor in ensuring treatment efficacy and patient safety. Especially for tumors that move with respiration, if the tumor cannot be accurately positioned, the radiation may miss the tumor target, resulting in the tumor cells not receiving a sufficient radiation dose and thus not being effectively killed. At the same time, it may also cause unnecessary damage to the surrounding healthy tissues.

Current radiotherapy systems with image-guided functionality acquire real-time image information of tumors through imaging apparatus (including a tube and an oppositely arranged detector) and perform tumor positioning and tracking (i.e., image guidance). However, the imaging apparatus is located in a fixed position in the radiotherapy system, which means that its field of view is limited and may only acquire tumor images from a fixed angle. At certain angles, such as when the tumor is overlappingly interfered with by other tissues such as the heart, spine, ribs, etc., the tumor may not be identified or distinguished, and thus the position of the tumor cannot be accurately positioned and tracked. In view of this, how to accurately position and track tumors is a problem that needs to be solved in the art.

In view of this, the tumor positioning method provided in the present disclosure first acquires a target projection image and a reference image of a target object containing a tumor at a target angle. Then, a tumor positioning manner that may position and identify the tumor at that angle is selected according to the target angle corresponding to the target projection image, and matching processing is performed on the target projection image and the reference image, to obtain the position of the tumor in the target projection image. In other words, the technical solutions may achieve tumor positioning and tracking for projection images at various angles using corresponding tumor positioning manners. Therefore, the technical solutions are not limited by the angle of the projection image when achieving tumor positioning, which may solve the problem of being unable to identify or distinguish the tumor at certain specific angles and thus failing to accurately position and track the position of the tumor, greatly improving the reliability of tumor positioning and identification.

The implementations of the embodiments of the present disclosure will be described in detail below with reference to the drawings in the specification.

It should be noted that, the embodiments of the present disclosure may be referenced or used in conjunction with each other. For example, the same or similar steps, method embodiments, system embodiments and apparatus embodiments may all be referred to each other without limitation.

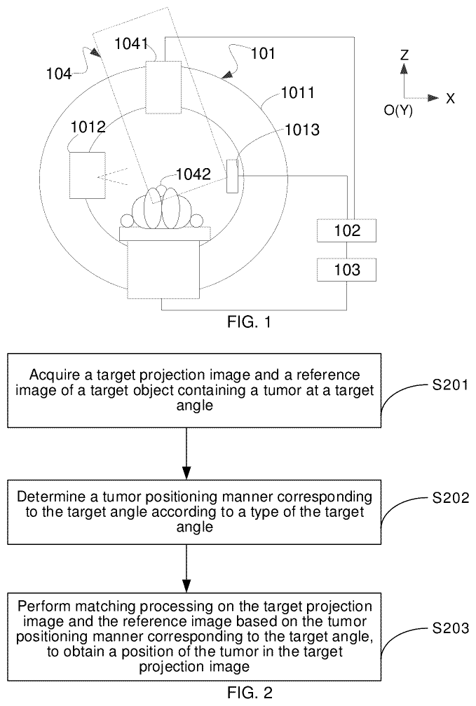

FIG. 1 is a schematic diagram of a scene of a radiotherapy system provided in the embodiments of the present disclosure and the radiotherapy system may include an image guidance radiotherapy device 101, an imaging computer device 102, a control apparatus 103, and a respiratory detection apparatus 104.

The image guidance radiotherapy device 101 is specifically explained below.

The image guidance radiotherapy device 101 may include a gantry 1011 and an image guidance apparatus disposed on the gantry 1011, where the image guidance apparatus includes a light source 1012 and a detector 1013. The light source 1012 is used to emit a light beam, and the detector 1013 is used to receive the light beam passing through a target object (patient), to generate a projection image of the target object.

In the embodiments of the present disclosure, the detector 1013 may be a flat-panel detector or a curved surface detector and a form of the detector 1013 is not specifically limited to the embodiments of the present disclosure.

In the embodiments of the present disclosure, the image guidance apparatus may be at least one of: a cone beam computed tomography (CBCT) apparatus, a computed tomography (CT) device, and a magnetic resonance (MR) device, that is, the image guidance apparatus may be a CBCT apparatus, a CT device, or an MR device, or may also include any two of the CBCT apparatus, the CT device, and the MR device, or the image guidance apparatus may further include the CBCT apparatus, the CT apparatus and the MR apparatus. The embodiments of the present disclosure do not specifically limit the form of the image guidance apparatus.

When the image guidance apparatus is the CBCT apparatus, the light source 1012 is an X-ray tube and the detector 1013 is a flat-panel detector.

In the embodiments of the present disclosure, the number of light sources 1012 and the number of detectors 1013 are not limited thereto. For example, the number of light sources 1012 may be one or more. Similarly, the number of detectors 1013 may be one or more. When both the number of light sources 1012 and number of detectors 1013 is multiple, two-dimensional images (i.e., projection images, also referred to as kilovoltage (KV) images) of multiple internal two-dimensional (2D) planes of the target subject may be generated at a certain gantry angle (or time point).

In some embodiments, when the number of light sources 1012 and the number of detectors 1013 is one, the light source and the detector may be positioned in the direction of the Z-axis as shown in FIG. 1. Thus, the position of the tumor of the target object in the X-axis direction and the Y-axis direction may be obtained, where the position of the tumor of the target object in the Y-axis direction represents the position of the tumor of the target object in a head-to-feet direction. The position of the tumor in the X-axis represents the position of the tumor of the target object in the left-right direction (the left and right of the target subject).

In some embodiments, when the number of light sources 1012 and the number of detectors 1013 is two, one set of light sources and detectors may be positioned in the direction of the Z-axis as shown in FIG. 1, to obtain the position of the tumor of the target object in the X-axis direction and Y-axis direction, and the other set of light sources and detectors may be positioned in the direction of the X-axis as shown in FIG. 1, to obtain the position of the tumor of the target object in the Z-axis direction and Y-axis direction. Thus, the three-dimensional spatial position of the tumor of the target object may be obtained based on the position of the tumor of the target object in the X-axis direction and Y-axis direction, and the position of the tumor of the target object in the Z-axis direction and Y-axis direction. The position of the tumor of the target object in the Z-axis direction represents the position of the tumor of the target object in the front-back (front and back of the target object, shown as up and down in FIG. 1) direction.

The frame 1011 may be a ring frame, a C-arm frame, a drum-shaped frame, a multi-layer bowl-shaped/tubular structure frame, etc., the frame 1011 is a rotating frame that may move around a rotation axis. When the frame 1011 rotates, the light source 1012 and the detector 1013 rotate around the Y-axis at any angle, so that a two-dimensional image (i.e., a projection image) of any 2D plane of the target object may be generated.

The respiration detection apparatus 104 is specifically explained below.

In some embodiments, the respiratory detection apparatus 104 is used to detect a respiratory signal of the target object.

In some embodiments, the respiratory detection apparatus 104 may include an optical camera 1041 and at least one optical marker 1042 disposed on a chest surface of the target object. Exemplarily, the optical camera 1041 may be an infrared camera, and correspondingly, the optical marker 1042 may be an infrared marker, or other types of optical cameras and matching optical markers. The form of the respiration detection apparatus 104 is not specifically limited to the embodiments of the present disclosure.

The imaging computer device 102 and the control apparatus 103 are explained below.

The imaging computer device 102 is communicatively connected to the control apparatus 103, the detector 1013, and the respiratory detection apparatus 104. The control apparatus 103 is communicatively connected to a support apparatus in the image guidance radiotherapy device 101 used to support the target object, and is used to control the movement of the support apparatus or control the image guidance radiotherapy device 101 to stop beam output based on a result of the image guidance.

In some embodiments, the imaging computer device 102 is a computer device with a graphical user interface (GUI), and the imaging computer device includes one or more processors, a memory, and one or more applications. Exemplarily, the imaging computer device 102 may include an image guidance system (IGS) application, the processor of the imaging computer device executes the IGS application to implement as follows: acquiring a target projection image and a reference image of a target object comprising a tumor at a target angle; determining a tumor positioning manner corresponding to the target angle according to a type of the target angle; and performing matching processing on the target projection image and the reference image based on the tumor positioning manner corresponding to the target angle, to obtain a position of the tumor in the target projection image.

In the embodiments of the present disclosure, the image computer device 102 and the control apparatus 103 may be independent servers, or may also be a server network or a server cluster composed of servers. For example, the computer devices described in the embodiments of the present disclosure include, but are not limited to, computers, network hosts, a single network server, multiple network server sets, or a cloud server composed of multiple servers. Herein, the cloud server is composed of a large number of computers or network servers based on cloud computing.

In the embodiments of the present disclosure, the imaging computer device 102 and the control apparatus 103 may also be general-purpose computer devices or special-purpose computer devices. In specific implementations, the computer device may be a desktop computer, a laptop computer, a network server, a personal digital assistant (PDA), a mobile phone, a tablet computer, a wireless terminal device, a communication device, an embedded device, etc., the type of computer device is not limited to the present embodiment.

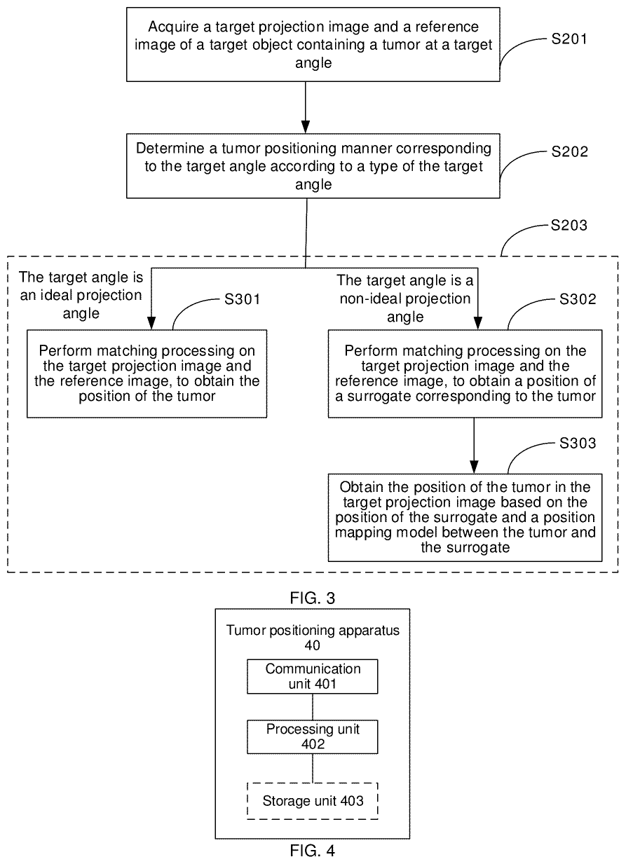

In some embodiments, the tumor positioning method shown in the embodiments of the present disclosure may be performed by the above-mentioned radiotherapy system. The tumor positioning method provided in the embodiments of the present disclosure is explained in detail below with reference to FIG. 2 and FIG. 3.

FIG. 2 is a flowchart of a tumor positioning method provided in the embodiments of the present disclosure. As shown in FIG. 2, the method includes the following S201 to S203.

In S201, a target projection image and a reference image of a target object containing a tumor at a target angle is acquired.

In some embodiments, the target projection image may also be referred to as a KV image. Herein, the target projection image is a two-dimensional image generated based on the internal projection of the target object (patient).

In some embodiments, the reference image may be a plan projection image or a setup projection image. Herein, the plan projection image is a two-dimensional image (e.g., a digital reconstructed radiograph (DRR) image) generated by digitally projecting the plan image of the target object at the target angle. The setup projection image is a two-dimensional projection image acquired when the target object is set up.

In some embodiments, the target angle is an ideal projection angle or a non-ideal projection angle. Herein, the ideal projection angle is an angle within a target angle range. The non-ideal projection angle is an angle outside the target angle range.

It should be noted that, in the projection image generated by imaging the target object within the target angle range, the tumor is not interfered with by other tissues or the degree of interference is small, for example, the degree of interference is less than or equal to a threshold. In the projection image generated by imaging the target object outside the target angle range, the degree of interference from other tissues on the tumor is greater than the threshold.

Furthermore, in the reference image generated by imaging the target object within the target angle range, the tumor is not interfered with by other tissues or the degree of interference is less than or equal to the threshold. In the reference image generated by imaging the target object outside the target angle range, the degree of interference from other tissues on the tumor is greater than the threshold.

In some embodiments, for the process for determining the target angle range, reference may be made to the Embodiment 1 below specifically, which will not be repeated herein.

In S202, a tumor positioning manner corresponding to the target angle is determined according to a type of the target angle.

In an implementation, the implementation process of S202 includes: in a case where the type of the target angle is a type of an ideal projection angle, that is, when the target angle is within the target angle range, determining the tumor position manner corresponding to the target angle as that: perform matching processing on the target projection image and the reference image, to directly determine a position of the tumor.

It should be noted that, in a case where the type of the target angle is the type of the ideal projection angle, the tumor in the target projection image is not overlappingly interfered with by other tissues (such as the heart, spinal column, ribs, etc.), and the tumor in the target projection image may be identified. Therefore, the target projection image has the capability to accurately position the tumor. Furthermore, in a case where the type of the target angle is the type of the ideal projection angle, the tumor in the reference image is not overlappingly interfered with by other tissues, and the tumor in the reference image may be identified. Therefore, this reference image has the capability to accurately position and track the position of the tumor. Therefore, in a case where the type of the target angle is the type of the ideal projection angle, by performing matching processing on the target projection image and the reference image, the positioning and tracking of the tumor in the target projection image may be realized.

In another implementation, the implementation process of S202 includes: in a case where the type of the target angle is a type of a non-ideal projection angle, that is, the target angle is not within the target angle range, determining the tumor positioning manner corresponding to the target angle as that: perform matching processing on the target projection image and the reference image, to determine a position of a surrogate corresponding to the tumor; and then indirectly realize the positioning and tracking of the tumor in the target projection image through the position of the surrogate.

Herein, the surrogate of the tumor may be any other surrogates except the tumor, such as a diaphragm, a lung wall, or a lung apex, etc. Thus, the object monitored in image guidance may be replaced by the surrogate instead of the tumor, and the motion of the surrogate is consistent with the motion of the tumor. Of course, the above description is only an exemplary description of the surrogates of the tumor, the surrogates of the tumor may also be human tissues that satisfy preset requirements for contrast and clarity in the image, and the present disclosure does not impose any limitations thereto.

It should be noted that, in a case where the type of the target angle is the type of the non-ideal projection angle, the tumor in the target projection image is overlappingly interfered with by other tissues (such as the heart, spinal column, ribs, etc.), while the position of the surrogate of the tumor is not interfered with, therefore, the tumor cannot be clearly identified in the target projection image, while the surrogate of the tumor may be clearly identified. Therefore, the target projection image does not have the conditions to accurately position the position of the tumor, but it does have the conditions to accurately position the position of the surrogate of the tumor. Furthermore, in a case where the type of the target angle is the type of the non-ideal projection angle, the tumor in the reference image is also overlappingly interfered with by other tissues, while the position of the surrogate of the tumor is not interfered with. Therefore, in the type of the target angle is the type of the non-ideal projection angle, the positioning and tracking of the tumor may be indirectly realized by determining the position of the surrogate corresponding to the tumor.

In S203, matching processing is performed on the target projection image and the reference image based on the tumor positioning manner corresponding to the target angle, to obtain a position of the tumor in the target projection image.

In an implementation, the implementation process of S203 includes: in a case where the type of the target angle is the type of the ideal projection angle, matching processing is performed on the target projection image and the reference image, to obtain the position of the tumor.

Specifically, for the above specific implementation process of obtaining the position of the tumor in a case where the type of the target angle is the type of the ideal projection angle, reference may be made to the embodiments shown in S301, which will not be repeated herein.

In another implementation, the implementation process of S203 further includes: in a case where the type of the target angle is the type of the non-ideal projection angle, performing matching processing on the target projection image and the reference image, to obtain the position of the surrogate corresponding to the tumor, and obtaining the position of the tumor in the target projection image based on the position of the surrogate and a position mapping model between the tumor and the surrogate.

Specifically, for the above specific implementation process of obtaining the position of the tumor in the target projection image in a case where the type of the target angle is the type of the non-ideal projection angle, reference may be made to the embodiments shown in S302, which will not be repeated herein.

Based on the above technical solutions, the tumor positioning method provided in the present disclosure, the target projection image and the reference image of the target object containing the tumor at the target angle are first acquired. Then, a tumor positioning manner that may position and identify the tumor at that angle is selected according to the target angle corresponding to the target projection image, and matching processing is performed on the target projection image and the reference image, to obtain the position of the tumor in the target projection image. In other words, the technical solutions may achieve tumor positioning and tracking for projection images at various angles using corresponding tumor positioning manners. Therefore, the technical solutions are not limited by the angle of the projection image when achieving tumor positioning. It can solve the problem that tumors cannot be identified or distinguished at certain specific angles, thus making it impossible to accurately locate and track the tumor's position, and greatly improves the reliability of tumor positioning and identification.

As an embodiment of the present disclosure, in combination with FIG. 2, as shown in FIG. 3, the implementation process of performing matching processing on the target projection image and the reference image, to obtain the position of the tumor in the target projection image in the above S203 may be implemented by the S301 or S302-S303 as follows.

In S301, in a case where the target angle is an ideal projection angle, matching processing is performed on the target projection image and the reference image, to obtain the position of the tumor.

In an implementation, in response to that the reference image is the plan projection image, the implementation process of S301 may include: performing registration processing on the target projection image and the plan projection image, to obtain the position of the tumor.

Herein, for the description of the plan projection image, reference is made to the embodiments shown in S201, which will not be repeated herein.

Furthermore, in response to that the reference image is the plan projection image, then the plan projection image within the target angle range is not interfered with by other tissues or the degree of interference is less than or equal to a threshold. In the plan projection image outside the target angle range, the degree of interference from other tissues is greater than the threshold.

It should be noted that, in a case where the target angle is the ideal projection angle, the tumor may be clearly shown in the plan projection image. Therefore, by performing registration processing on the plan projection image and the target projection image, the position of the tumor in the target projection image may be determined.

In another implementation, in a case where to the reference image is a setup projection image, the implementation process of S301 may include: performing matching processing on the tumor in the target projection image based on a contour of the tumor in the setup projection image, to obtain the position of the tumor in the target projection image.

Herein, for the description of the setup projection image, reference is made to the embodiments shown in S201, which will not be repeated herein.

It should be noted that, in a case where the target angle is the ideal projection angle, the setup projection image has already outlined the contour of the tumor. Therefore, matching processing is performed on the tumor in the target projection image, to obtain the contour of the tumor in the target projection image, and then the position of the tumor may be determined based on the contour of the tumor in the target projection image.

In S302, in a case where the target angle is a non-ideal projection angle, matching processing is performed on the target projection image and the reference image, to obtain a position of a surrogate corresponding to the tumor.

Herein, the non-ideal projection angle is an angle outside the target angle range.

In an implementation, in response to that the reference image is the plan projection image, the implementation process of S302 may include: performing registration processing on the target projection image and the plan projection image, to obtain the position of the surrogate corresponding to the tumor.

It should be noted that, in a case where the target angle is the non-ideal projection angle, the tumor in the plan projection image is interfered with by other tissues and is not clearly shown, but the surrogate corresponding the tumor is clearly shown. Therefore, by performing registration processing on the plan projection image and the target projection image, position information of the surrogate in the target projection image may be determined.

In another implementation, in a case where to the reference image is the setup projection image, the implementation process of S302 may include: performing matching processing on the surrogate in the target projection image based on a contour of the surrogate in the setup projection image, to obtain the position of the surrogate in the target projection image.

It should be noted that, in a case where the target angle is the non-ideal projection angle, the setup projection image has already outlined the contour of the surrogate corresponding to the tumor. Therefore, matching processing is performed on the surrogate in the target projection image based on the contour of the surrogate in the setup projection image, to obtain the position of the surrogate in the target projection image.

In S303, the position of the tumor in the target projection image is obtained based on the position of the surrogate and a position mapping model between the tumor and the surrogate.

It can be understood that, since the position mapping model between the tumor and surrogate contains a mapping relationship between a position trajectory of the tumor and a position trajectory of the surrogate, by inputting the position information of the surrogate into the position mapping model between the tumor and the surrogate, position information of the tumor corresponding to the position information of the surrogate may be obtained through mapping, that is, the position of the tumor in the target projection image.

Based on the above technical solutions, in a case where the target angle is the ideal projection angle, matching processing is performed on the target projection image and the reference image, to obtain the position of the tumor directly. In a case where the target angle is the non-ideal projection angle, the position of the tumor cannot be directly identified due to the degree of interference with the tumor in both the projection image and the reference image. In the present disclosure, the position of the surrogate corresponding to the tumor is obtained by performing matching processing on the target projection image and the reference image, and the position of the tumor in the target projection image is obtained based on the position of the surrogate and the position mapping model between the tumor and the surrogate, which greatly enhances the reliability of tumor position identification.

Regarding the target angle range, the position mapping model between the tumor and the surrogate, and the position of the tumor in the above embodiments, in the following, the implementation process of determining the target angle range is specifically described through Embodiment 1; a process of establishing the position mapping model between the tumor and surrogate is specifically described through Embodiment 2; the implementation process of establishing a respiratory motion model of the target object using the position of the tumor is specifically described through Embodiment 3; and a process of updating position mapping model between the tumor and surrogate established in Embodiment 2 and/or performing updating processing on the respiratory motion model of the target object established in Example 3 is specifically described through Embodiment 4.

### Embodiment 1

In some embodiments, the implementation process of determining the target angle range may include S11-S12 as follows.

In S11, plan projection images of a tumor at multiple projection angles are acquired.

Herein, the plan projection image is a two-dimensional image (such as a DRR image) generated by digitally projecting the plan image (e.g., CT image or MRI image) of the target object at a projection angle.

It should be noted that, since the clarity of the tumor and other tissues in the plan projection image is greater than a preset clarity threshold, the degree of interference on the tumor in the plan projection image may be directly determined. Furthermore, it facilitates that division is performed on the multiple projection angles based on the plan projection images at multiple projection angles subsequently, thereby obtaining the target angle range.

In S12, the target angle range is acquired from the multiple projection angles based on the plan projection images at the multiple projection angles.

In an implementation, for the plan projection image at each projection angle, the degree of the interference on the tumor in the plan projection image is determined. If the tumor is not interfered with or the degree of the interference is less than or equal to the threshold, it means that the contrast and clarity of the tumor in the target projection image at the projection angle satisfy a preset requirement, and thus the projection angle of the plan projection image is classified into the target angle range.

Conversely, if the tumor is interfered with or the degree of the interference is greater than the threshold, the projection angle of the plan projection image is classified as an interference angle range (that is, an angle range outside the target angle range).

In some embodiments, the implementation process of determining the degree of interference on the tumor includes: determining a proportion of an area of the tumor interfered with by other tissues to a total area of the tumor; and taking this proportion as the degree of interference on the tumor.

Based on the above technical solutions, plan projection images of the tumor at the multiple projection angles are acquired to simulate a path of rays passing through the tumor. Then, based on the plan projection images at the multiple projection angles, the target angle range is acquired from the multiple projection angles.

Exemplarily, a maximum angle range may be [0 degrees, 360 degrees], and there may be multiple target angle ranges, such as [0 degrees, 30 degrees], [180 degrees, 210 degrees]. There is no overlap between multiple target angle ranges and other angle ranges except the target angle range are interference angle ranges, such as (30 degrees, 180 degrees) and (210 degrees, 360 degrees).

### Embodiment 2

As an embodiment of the present disclosure, the process of establishing the position mapping model between the tumor and the surrogate may be implemented through the S21-S23 as follows.

In S21, multiple projection images of the target object within the target angle range are acquired.

Herein, each projection image (e.g., each KV image in a sequence of KV images) of the multiple projection images includes the tumor and the surrogate.

Herein, for the description of the surrogate of the tumor, reference is made to the description of the surrogate of the tumor in the embodiments shown in S202, which will not be repeated herein.

It should be noted that, within the target angle range, the tumor and the surrogate in the projection image are not interfered with by other tissues or the degree of interference is less than or equal to the threshold; while outside the target angle range, the degree of interference on the tumor and the surrogate by other tissues is greater than the threshold.

In other words, within the target angle range, the clarity of the position of the tumor and the position of the surrogate in the projection image satisfies a preset clarity threshold. While, outside the target angle range, the clarity of the position of the tumor and the position of the surrogate in the projection does not satisfy the preset clarity threshold. Therefore, acquiring multiple projection images of the target object within the target angle range may facilitate the determination of the mapping relationship between the position of the tumor and the position of the surrogate according to the position of the tumor in the projection images subsequently.

In S22, a position trajectory of the tumor and a position trajectory of the surrogate are determined based on the multiple projection images.

Herein, the position trajectory of the tumor is used to indicate changes in the position of the tumor, and the position trajectory of the surrogate is used to indicate changes in the position of the surrogate.

In an implementation, the implementation process of above S22 includes: serially recording the positions of the tumor in the multiple projection images according to an acquisition order of the projection image, to obtain the position trajectory of the tumor. The positions of the surrogate in multiple projection images are recorded serially according to the acquisition order of the projection images, to obtain the position trajectory of the surrogate.

In S23, a position mapping model between the tumor and the surrogate is established based on the position trajectory of the tumor and the position trajectory of the surrogate.

In an implementation, the position trajectory of the tumor and the position trajectory of the surrogate are mapped and combined by a preset mapping method (such as linear mapping, non-linear mapping, machine learning algorithm, etc.), to construct the position mapping model between the tumor and the surrogate.

It should be noted that, S21-S23 in Embodiment 2 may be performed before treatment so that the position mapping model may be used to position the tumor during the subsequent treatment process.

Based on the above technical solutions, the multiple projection images of the target object within the target angle range are acquired. Based on the multiple projection images, the position trajectory of the tumor and the position trajectory of the surrogate are determined. Furthermore, based on the position trajectory of the tumor and the position trajectory of the surrogate, the position mapping model between the tumor and the surrogate is established. This model has strong correlation and stability, which facilitates inferring the position of the tumor by using this position mapping model and the position of the surrogate when performing tumor positioning for the target projection images acquired under non-ideal projection angles, thereby achieving tumor positioning.

### Embodiment 3

In some embodiments, the implementation process of establishing the respiratory motion model of the target object includes: establishing the respiratory motion model of the target object based on the position of the tumor in the target projection image and a respiratory signal of the target object.

Herein, the respiratory motion model of the target object is used to reflect the mapping relationship between the position of the tumor and the respiratory signal.

It should be noted that, the respiratory motion model is obtained by combining the positions of the tumor under the ideal projection angle and the positions of the tumor under the non-ideal projection angle, with the respiratory signal. In other words, the respiratory motion model includes the positions of the tumor under various angles. Therefore, the tumor positioning may also be implemented by inputting respiratory data of the target object into the respiratory motion model.

### Embodiment 4

As an embodiment of the present disclosure, the present disclosure may also perform updating processing on the position mapping model between the tumor and the surrogate established in Embodiment 2 above, and/or perform updating processing on the respiratory motion model of the target object established in Embodiment 3 above.

In some embodiments, a position offset of the tumor in the target projection image is determined, and in response to that the position offset is not within a threshold range, the position mapping model between the tumor and the surrogate, and/or the respiratory motion model of the tumor are updated based on the position of the tumor in the target projection image.

Herein, the position offset of the tumor is an offset between the position of the tumor position in the target projection image and a mapping position of the tumor obtained through the respiratory motion model; the mapping position of the tumor is the position of the tumor obtained by mapping the respiratory signal corresponding to the target angle into the respiratory motion model.

In an implementation, when the target projection image is acquired at the target angle, the respiratory signal corresponding to the target angle is input into the respiratory motion model, to obtain the position of the tumor through mapping. Then, the offset between the mapped position of the tumor and the position of the tumor in the target projection image is determined. If the offset is not within the threshold range, it indicates that the offset of the position of the tumor obtained based on the respiratory motion model is too large, and the respiratory motion model needs to be updated. Furthermore, based on the position of the tumor in the target projection image, the respiratory motion model of the tumor and the position mapping model between the tumor and the surrogate are updated, to ensure the reliability of the respiratory motion model and the position mapping model between the tumor and the surrogate.

In some other embodiments, in response to that the type of the target angle is the type of the ideal projection angle, after performing matching processing on the target projection image and the reference image to directly obtain the position of the tumor, the position of the tumor is used to check and update the position mapping model between the tumor and the surrogate and the respiratory motion model. In response to that the type of the target angle is the type of the non-ideal projection angle, after indirectly obtaining the position of the tumor by using the position of the surrogate of the tumor, the position of the tumor is used to check and update the respiratory motion model.

The embodiments of the present disclosure may divide an electronic device into functional modules or functional units according to the above method examples; for example, various functional modules or functional units may be divided corresponding to various functions, or two or more functions may also be integrated into a processing module. The above integrated module may be implemented in the form of hardware or may be implemented in the form of a software functional module or functional unit. Herein, the division of the modules or units in the embodiments of the present disclosure is schematic, which is only a logical functional division, and there may be other divisions in actual implementations.

As shown in FIG. 4, FIG. 4 is a structural schematic diagram of a tumor positioning apparatus 40 provided in the embodiments of the present disclosure, and the tumor positioning apparatus 40 includes a communication unit 401 and a processing unit 402.

The communication unit 401 is configured to, acquire a target projection image and a reference image of a target object containing a tumor at a target angle; determine a tumor positioning manner corresponding to the target angle according to a type of the target angle; and perform matching processing on the target projection image and the reference image based on the tumor positioning manner corresponding to the target angle, to obtain a position of the tumor in the target projection image.

In an implementation, the processing unit 402 is specifically configured to: in response to that the target angle is an ideal projection angle, perform matching processing on the target projection image and the reference image, to obtain the position of the tumor; where the ideal projection angle is an angle within a target angle range; or in response to that the target angle is a non-ideal projection angle, perform matching processing on the target projection image and the reference image, to obtain a position of a surrogate corresponding to the tumor; and obtain the position of the tumor in the target projection image based on the position of the surrogate and a position mapping model between the tumor and the surrogate; where the non-ideal projection angle is an angle outside the target angle range.

In an implementation, the processing unit 402 is specifically configured to: in response to that to the reference image is a plan projection image, perform registration processing on the target projection image and the plan projection image, to obtain the position of the tumor; where the plan projection image is a two-dimensional image generated by digitally projecting a plan image of the target object at the target angle; and in response to that the reference image is a setup projection image, perform matching processing on the tumor in the target projection image based on a contour of the tumor in the setup projection image, to obtain the position of the tumor in the target projection image; where the setup projection image is a two-dimensional projection image acquired when the target object is set up.

In an implementation, the processing unit 402 is specifically configured to: in response to that the reference image is a plan projection image, perform registration processing on the target projection image and the plan projection image, to obtain the position of the surrogate corresponding to the tumor; and in response to that the reference image is a setup projection image, perform matching processing on the surrogate in the target projection image based on a contour of the surrogate in the setup projection image, to obtain the position of the surrogate in the target projection image.

In an implementation, the processing unit 402 is specifically configured to: acquire plan projection images of the tumor at multiple projection angles; where the plan projection image is a two-dimensional image generated by digitally projecting a plan image of the target object at the projection angle; and acquire a target angle range from the multiple projection angles based on the plan projection images at the multiple projection angles.

In an implementation, the communication unit 401 is further configured to, acquire multiple projection images of the target object within the target angle range, where each projection image of the multiple projection images includes the tumor and the surrogate; determine a position trajectory of the tumor and a position trajectory of the surrogate based on the multiple projection images, where the position trajectory of the tumor and the position trajectory of the surrogate respectively indicate a position change of the tumor and a position change of the surrogate; and establish the position mapping model between the tumor and the surrogate based on the position trajectory of the tumor and the position trajectory of the surrogate.

In an implementation, the processing unit 402 is further configured to: establish a respiratory motion model of the target object based on the position of the tumor in the target projection image and a respiratory signal of the target object; where the respiratory motion model of the target object is used to reflect a mapping relationship between the position of the tumor and the respiratory signal.

In an implementation, the processing unit 402 is further configured to: determine a position offset of the tumor in the target projection image, and in response to that the position offset is not within a threshold range, update the position mapping model between the tumor and the surrogate, and/or update the respiratory motion model of the target object based on the position of the tumor in the target projection image; and where the position offset of the tumor is an offset between the position of the tumor in the target projection image and a mapped position of the tumor obtained by the respiratory motion model; and the mapped position of the tumor is a position of the tumor obtained through mapping after inputting a respiratory signal corresponding to the target angle into the respiratory motion model.

In an implementation, the tumor positioning apparatus 40 may further include a storage unit 403 (shown in a dashed box in FIG. 4), the storage unit 403 stores a program or instructions and the program or the instructions, when executed on the processing unit 402, cause the tumor positioning apparatus 40 to perform the tumor positioning method described in the above method embodiments.

When implemented in hardware, the embodiments of the present disclosure also provide an electronic device for performing the tumor positioning method shown in the above method embodiments.

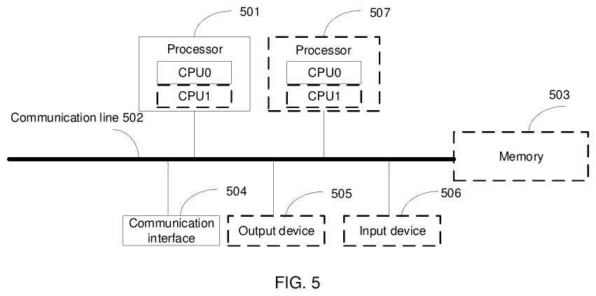

Specifically, FIG. 5 is a schematic diagram of a hardware structure of an electronic device, provided by the embodiments of the present disclosure. As shown in FIG. 5, the electronic device includes at least one processor 501, a communication line 502, at least one communication interface 504, and a memory 503 configured to store processor-executable instructions. Herein, the processor 501, the memory 503 and the communication interface 504 may be connected through the communication line 502. The processor is configured to execute instructions to implement the tumor positioning method described in the method embodiments of the present disclosure.

The processor 501 may be a central processing unit (CPU), or an application specific integrated circuit (ASIC), or one or more integrated circuits configured to implement the embodiments of the present disclosure, e.g., one or more digital signal processors (DSPs), or one or more field programmable gate arrays (FPGAs).

The communication line 502 may include a communication path for communicating information between the above components.

The communication interface 504 is configured to communicate with other devices or communication networks and may use any transceiver-type apparatus, such as Ethernet, radio access network (RAN), wireless local area networks (WLAN), etc.

The memory 503 may be a read-only memory (ROM) or other types of static storage devices that may store static information and instructions, or a random access memory (RAM) or other types of dynamic storage device that may store information and instructions, or may be an electrically erasable programmable read-only memory (EEPROM), a compact disc read-only memory (CD-ROM) or other optical disc storages, optical disc storage (including compact optical disc, laser disc, optical disc, digital versatile disc, Blu-ray disc, or the like), a magnetic disk storage medium or other magnetic storage devices, or any other medium capable of including or storing desired program codes in the form of instructions or data structures and being accessed by a computer, which is not limited thereto.

In a design, the memory 503 may exist independently of the processor 501, i.e., the memory 503 may be an external memory of the processor 501. In this case, the memory 503 may be connected to the processor 501 through the communication line 502, and the memory 103 is configured for storing execution instructions or application codes, and is controlled and executed by the processor 501, to implement the tumor positioning method provided in the following embodiments of the present disclosure. In another design, the memory 503 may also be integrated with the processor 501, that is, the memory 503 may be an internal memory of the processor 501. For example, the memory 503 may be a cache and may be configured to temporarily store some data and instruction information.

As an implementation, the processor 501 may include one or more CPUs, such as CPU0 and CPU1 in FIG. 5. As another implementation, the electronic device may include multiple processors, such as the processor 501 and processor 507 in FIG. 5.

As yet another implementation, the electronic device may also include an output device 505, such as various types of displays, speakers, etc.; and an input device 506, such as a keyboard, mice, etc.

From the above description of the implementation, those skilled in the art may clearly understand that for the convenience and simplicity of description, only the division of the above respective functional modules is used as an example for explanation. In practical applications, the above functions may be allocated to be completed by different functional modules according to requirements, i.e., internal structures of the apparatus may be divided into different functional modules, to complete all or a part of the functions described above. As for the specific working processes of the above-described system, apparatus, and unit, reference may be made to the corresponding processes in the above method embodiments, which are not repeated herein.

The embodiments of the present disclosure provide a computer program product including computer instructions, and the computer program product, when executed on a computer, causes the computer to perform the tumor positioning method described in the above method embodiments.

The embodiments of the present disclosure further provide a computer readable storage medium and the computer readable storage medium has stored instructions therein, and the instructions, when executed on a computer, cause the computer to perform the tumor positioning method in the method flow shown in the above method embodiments.

Herein, the computer readable storage medium may be, for example, but is not limited to, electronic, magnetic, optical, electromagnetic, infrared, or semiconductor systems, apparatuses or devices, or any combination thereof. More specific examples (non-exhaustive list) of the computer readable storage medium include: an electrical connection with one or more wires, a portable computer disk, a hard drive, a random access memory (RAM), a read-only memory (ROM), an erasable programmable read only memory (EPROM), a register, a hard disk, an optical fiber, a portable compact disc read-only memory (CD-ROM), an optical storage device, a magnetic storage device, or any suitable combination of the above, or any other forms of computer readable storage media well known in the art. An exemplary storage medium is coupled to a processor, enabling the processor to read information from the storage medium and write information to the storage medium. Of course, the storage media may also be a component of the processor. The processor and the storage media may be located in an application specific integrated circuit (ASIC). In the embodiments of the present disclosure, the computer readable storage medium may be any tangible medium that contains or stores a program for use by or in connection with an instruction execution system, an apparatus, or a device.

The various implementations of systems and technologies described above in the present disclosure may be implemented in a digital electronic circuit system, an integrated circuit system, a field programmable gate array, an application specific integrated circuit, an application specific standard parts (ASSP), a system on chip (SOC) systems, a complex programmable logic device (CPLD), a computer hardware, a firmware, a software, and/or any combination thereof. These various implementations may include: being implemented in one or more computer programs and the one or more computer programs may be executed and/or interpreted on a programmable system including at least one programmable processor, the programmable processor may be a dedicated or general-purpose programmable processor, and may receive data and instructions from the storage system, at least one input apparatus, and at least one output apparatus, and transmit the data and the instructions to the storage system, the at least one input apparatus, and the at least one output apparatus.

The program code for implementing the method in the present disclosure may be written in any combination of one or more programming languages. These program codes may be provided to a processor or a controller of a general-purpose computer, a dedicated computer, or other programmable data processing apparatuses, so that the program code, when executed by the processor or the controller, enables the functions/operations specified in the flow chart and/or block diagram to be implemented. The program codes may be completely executed on the machine, partially executed on the machine, as an independent software package partially executed on the machine, and partially executed on remote machines or completely executed on remote machines or servers.

To provide interaction with a user, the systems and techniques described herein may be implemented on a computer, and the computer has: a display apparatus for displaying information to the user, such as a cathode ray tube (CRT) or a liquid crystal display (LCD) monitor; and a keyboard and a pointing apparatus (e.g., a mouse or trackball) through which the user provides input to the computer. Other types of apparatuses may also be used to provide interaction with the user; for example, the feedback provided to the user may be any form of sensory feedback (e.g., visual feedback, auditory feedback, or tactile feedback); and input from the user may be received in any form (including acoustic input, voice input, or tactile input).

The system and technology described herein may be implemented in a computing system that includes a background component (e.g., as a data server), or in a computing system that includes a middleware component (e.g., an application server), or in a computing system that includes a front-end component (e.g., a user computer with a graphical user interface or web browser through which users may interact with the implementation of the system and technology described herein), or in a computing system that includes any combination of the background component, the middleware component, or the front-end component. The components of the system may be interconnected through any form or medium of digital data communication (e.g., a communication network). Examples of the communication network include: a local area network (LAN), a wide area network (WAN), or the Internet.

A computer system may include a client and a server. The client and server are generally far away from each other and typically interact through the communication network. A client-server relationship is generated by running computer programs on respective computers that have client-server relationships with each other. The server may be a cloud server, a distributed system server, or a server that combines block chain.

Since the electronic devices, computer readable storage media, and computer program products in the embodiments of the present disclosure may be applied to the above methods, the technical effects they can achieve can also be referred to the above method embodiments. The embodiments of the present disclosure will not be repeated herein.

In several embodiments provided in the present disclosure, it will be understood that the disclosed system, device and method may be implemented through other manners. For example, the device embodiments described above are merely exemplary, for example, the division of the units is only a logical functional division. In actual implementation, there are other division manners, for example, a plurality of units or components are combined or integrated into another system, or some features may be ignored or not executed. Furthermore, the mutual coupling or direct coupling or communication connection shown or discussed may be an indirect coupling or communication connection through some interfaces, devices or units, and may be in an electrical, mechanical or other forms.

It should be understood that, various forms of processes shown in the above may be reordered, added, or deleted. For example, the respective steps described in the present disclosure may be performed in parallel, in sequence, or in different orders as long as the expected results of the technical solutions related to the present disclosure may be achieved, which is not be limited herein.

The specific implementations do not limit the scope of the present disclosure. Those skilled in the art should understand that various modifications, combinations, sub-combinations, and substitutions may be made according to design requirements and other factors. Any modifications, equivalent substitutions, and improvements made within the spirit and the principles of the present disclosure shall be included within the scope of the present disclosure.

The units described as separate components may or may not be physically separated; and the components used as units may or may not be physical units, that is, they may be located in one place, or may be distributed to multiple network units. Some or all of the units may be selected according to actual needs to achieve the purposes of the solutions of the present embodiments.

In addition, various functional units in various embodiments of the present disclosure may be integrated in a single processing unit, or each unit may physically exist separately, or two or more units may be integrated in a single unit.

The above are only specific implementations of the present disclosure, but the scope of protection of the present disclosure is not limited thereto, and any person skilled in the art may conceive of variations or replacements within the technical scope of the present disclosure, which shall fall within the protection scope of the present disclosure. Therefore, the protection scope of the present disclosure shall be subject to the protection scope of the claims.

What is claimed is:

1. A tumor positioning method, wherein the method comprises:
acquiring a target projection image and a reference image of a target object comprising a tumor at a target angle;
determining a tumor positioning manner corresponding to the target angle according to a type of the target angle; and
performing matching processing on the target projection image and the reference image based on the tumor positioning manner corresponding to the target angle, to obtain a position of the tumor in the target projection image.

2. The method according to claim 1, wherein performing matching processing on the target projection image and the reference image based on the tumor positioning manner corresponding to the target angle, to obtain the position of the tumor in the target projection image comprises:
in response to that the target angle is an ideal projection angle, performing matching processing on the target projection image and the reference image, to obtain the position of the tumor; wherein the ideal projection angle is an angle within a target angle range; or
in response to that the target angle is a non-ideal projection angle, performing matching processing on the target projection image and the reference image, to obtain a position of a surrogate corresponding to the tumor; and obtaining the position of the tumor in the target projection image based on the position of the surrogate and a position mapping model between the tumor and the surrogate; wherein the non-ideal projection angle is an angle outside the target angle range.

3. The method according to claim 2, wherein in response to that the target angle is the ideal projection angle, performing matching processing on the target projection image and the reference image, to obtain the position of the tumor comprises:
in response to that to the reference image is a plan projection image, performing registration processing on the target projection image and the plan projection image, to obtain the position of the tumor; wherein the plan projection image is a two-dimensional image generated by digitally projecting a plan image of the target object at the target angle; and
in response to that the reference image is a setup projection image, performing matching processing on the tumor in the target projection image based on a contour of the tumor in the setup projection image, to obtain the position of the tumor in the target projection image; wherein the setup projection image is a two-dimensional projection image acquired when the target object is set up.

4. The method according to claim 2, wherein in response to that the target angle is the non-ideal projection angle, performing matching processing on the target projection image and the reference image, to obtain the position of the surrogate corresponding to the tumor comprises:
in response to that the reference image is a plan projection image, performing registration processing on the target projection image and the plan projection image, to obtain the position of the surrogate corresponding to the tumor; and
in response to that the reference image is a setup projection image, performing matching processing on the surrogate in the target projection image based on a contour of the surrogate in the setup projection image, to obtain the position of the surrogate in the target projection image.

5. The method according to claim 2, wherein the method further comprises:
acquiring plan projection images of the tumor at multiple projection angles; wherein a plan projection image is a two-dimensional image generated by digitally projecting a plan image of the target object at the projection angle; and
acquiring a target angle range from the multiple projection angles based on the plan projection images at the multiple projection angles.

6. The method according to claim 2, wherein a process for establishing the position mapping model between the tumor and the surrogate comprises:
acquiring multiple projection images of the target object within the target angle range, wherein each projection image of the multiple projection images comprises the tumor and the surrogate;
determining a position trajectory of the tumor and a position trajectory of the surrogate based on the multiple projection images, wherein the position trajectory of the tumor and the position trajectory of the surrogate respectively indicate a position change of the tumor and a position change of the surrogate; and
establishing the position mapping model between the tumor and the surrogate based on the position trajectory of the tumor and the position trajectory of the surrogate.

7. The method according to claim 1, wherein the method further comprises:
establishing a respiratory motion model of the target object based on the position of the tumor in the target projection image and a respiratory signal of the target object; wherein the respiratory motion model of the target object is used to reflect a mapping relationship between the position of the tumor and the respiratory signal.

8. The method according to claim 1, wherein the method further comprises:
determining a position offset of the tumor in the target projection image, and in response to that the position offset is not within a threshold range, updating the position mapping model between the tumor and the surrogate, or updating the respiratory motion model of the target object or updating the position mapping model between the tumor and the surrogate and updating the respiratory motion model of the target object based on the position of the tumor in the target projection image; and
wherein the position offset of the tumor is an offset between the position of the tumor in the target projection image and a mapped position of the tumor obtained by the respiratory motion model; and the mapped position of the tumor is a position of the tumor obtained through mapping after inputting a respiratory signal corresponding to the target angle into the respiratory motion model.

9. An electronic device, wherein the electronic device comprises:
a processor; and
a memory configured to store instructions executable for the processor;
wherein the processor is configured to execute the instructions, to implement operations of:
acquiring a target projection image and a reference image of a target object comprising a tumor at a target angle;
determining a tumor positioning manner corresponding to the target angle according to a type of the target angle; and
performing matching processing on the target projection image and the reference image based on the tumor positioning manner corresponding to the target angle, to obtain a position of the tumor in the target projection image.

10. The device according to claim 9, wherein the processor is further configured to:
in response to that the target angle is an ideal projection angle, perform matching processing on the target projection image and the reference image, to obtain the position of the tumor; wherein the ideal projection angle is an angle within a target angle range; or
in response to that the target angle is a non-ideal projection angle, perform matching processing on the target projection image and the reference image, to obtain a position of a surrogate corresponding to the tumor; and obtain the position of the tumor in the target projection image based on the position of the surrogate and a position mapping model between the tumor and the surrogate; wherein the non-ideal projection angle is an angle outside the target angle range.

11. The device according to claim 10, wherein the processor is further configured to:
in response to that to the reference image is a plan projection image, perform registration processing on the target projection image and the plan projection image, to obtain the position of the tumor; wherein the plan projection image is a two-dimensional image generated by digitally projecting a plan image of the target object at the target angle; and
in response to that the reference image is a setup projection image, perform matching processing on the tumor in the target projection image based on a contour of the tumor in the setup projection image, to obtain the position of the tumor in the target projection image; wherein the setup projection image is a two-dimensional projection image acquired when the target object is set up.

12. The device according to claim 10, wherein the processor is further configured to:
in response to that the reference image is a plan projection image, perform registration processing on the target projection image and the plan projection image, to obtain the position of the surrogate corresponding to the tumor; and
in response to that the reference image is a setup projection image, perform matching processing on the surrogate in the target projection image based on a contour of the surrogate in the setup projection image, to obtain the position of the surrogate in the target projection image.

13. The device according to claim 10, wherein the processor is further configured to:
acquire plan projection images of the tumor at multiple projection angles; wherein the plan projection image is a two-dimensional image generated by digitally projecting a plan image of the target object at the projection angle; and
acquire a target angle range from the multiple projection angles based on the plan projection images at the multiple projection angles.

14. The device according to claim 10, wherein the processor is further configured to:
acquire multiple projection images of the target object within the target angle range, wherein each projection image of the multiple projection images comprises the tumor and the surrogate;
determine a position trajectory of the tumor and a position trajectory of the surrogate based on the multiple projection images, wherein the position trajectory of the tumor and the position trajectory of the surrogate respectively indicate a position change of the tumor and a position change of the surrogate; and
establish the position mapping model between the tumor and the surrogate based on the position trajectory of the tumor and the position trajectory of the surrogate.

15. The device according to claim 9, wherein the processor is further configured to:
establish a respiratory motion model of the target object based on the position of the tumor in the target projection image and a respiratory signal of the target object; wherein the respiratory motion model of the target object is used to reflect a mapping relationship between the position of the tumor and the respiratory signal.

16. The device according to claim 9, wherein the processor is further configured to:
determine a position offset of the tumor in the target projection image, and in response to that the position offset is not within a threshold range, update the position mapping model between the tumor and the surrogate, or update the respiratory motion model of the target object or update the position mapping model between the tumor and the surrogate and updating the respiratory motion model of the target object based on the position of the tumor in the target projection image; and
wherein the position offset of the tumor is an offset between the position of the tumor in the target projection image and a mapped position of the tumor obtained by the respiratory motion model; and the mapped position of the tumor is a position of the tumor obtained through mapping after inputting a respiratory signal corresponding to the target angle into the respiratory motion model.

17. A non-transitory computer readable storage medium, wherein the computer readable storage medium has stored instructions therein, and the instructions, when executed on a computer, so that the computer performs operations of:
acquiring a target projection image and a reference image of a target object comprising a tumor at a target angle;
determining a tumor positioning manner corresponding to the target angle according to a type of the target angle; and
performing matching processing on the target projection image and the reference image based on the tumor positioning manner corresponding to the target angle, to obtain a position of the tumor in the target projection image.

18. The storage medium according to claim 17, wherein the computer is further configured to:
in response to that the target angle is an ideal projection angle, perform matching processing on the target projection image and the reference image, to obtain the position of the tumor; wherein the ideal projection angle is an angle within a target angle range; or
in response to that the target angle is a non-ideal projection angle, perform matching processing on the target projection image and the reference image, to obtain a position of a surrogate corresponding to the tumor; and obtain the position of the tumor in the target projection image based on the position of the surrogate and a position mapping model between the tumor and the surrogate; wherein the non-ideal projection angle is an angle outside the target angle range.

19. The storage medium according to claim 18, wherein the computer is further configured to:
in response to that to the reference image is a plan projection image, perform registration processing on the target projection image and the plan projection image, to obtain the position of the tumor; wherein the plan projection image is a two-dimensional image generated by digitally projecting a plan image of the target object at the target angle; and
in response to that the reference image is a setup projection image, perform matching processing on the tumor in the target projection image based on a contour of the tumor in the setup projection image, to obtain the position of the tumor in the target projection image; wherein the setup projection image is a two-dimensional projection image acquired when the target object is set up.

20. The storage medium according to claim 18, wherein the computer is further configured to:
in response to that the reference image is a plan projection image, perform registration processing on the target projection image and the plan projection image, to obtain the position of the surrogate corresponding to the tumor; and
in response to that the reference image is a setup projection image, perform matching processing on the surrogate in the target projection image based on a contour of the surrogate in the setup projection image, to obtain the position of the surrogate in the target projection image.
